(19) United States
(12) Patent Application Publication
(10) Pub. No.: US 2026/0191415 A1
(43) Pub. Date: Jul. 9, 2026

(54) ERGODIC RELAY AND TIME REVERSAL FOR CALIBRATION-FREE HARDWARE PHOTOACOUSTIC IMAGE RECONSTRUCTION AND ULTRASONIC THERAPY

(71) Applicant: California Institute of Technology, Pasadena, CA (US)

(72) Inventor: Lihong Wang, Arcadia, CA (US)

(73) Assignee: California Institute of Technology, Pasadena, CA (US)

(21) Appl. No.: 19/379,417

(22) Filed: Nov. 4, 2025

Related U.S. Application Data
(60) Provisional application No. 63/716,044, filed on Nov. 4, 2024.

(51) Int. Cl. A61B 5/00; G01N 21/17; G01S 7/52; G01S 15/89

(52) U.S. Cl. CPC A61B 5/0095; G01N 21/1702; G01S 7/52046; G01S 15/8968; A61B 2562/046

(57) ABSTRACT

In some cases, photoacoustic imaging techniques involve detecting photoacoustic signals through an ergodic relay, emitting time-reversed photoacoustic signals back through the ergodic relay to an acoustic medium where acoustic radiation pressure causes surface displacements, optically imaging the surface profile of the acoustic medium at different heights, and constructing a 3D image of the object based on the surface profile at different heights.

### CROSS-REFERENCE TO RELATED APPLICATION

This application claims benefit of and priority to U.S. Provisional Patent Application No. 63/716,044, titled “Ergodic Relay and Time Reversal for Calibration-Free Hardware Photoacoustic Image Reconstruction and Ultrasonic Therapy,” and filed on Nov. 4, 2024, which is incorporated by reference herein in its entirety and for all purposes.

### FIELD

Certain aspects relate generally to photoacoustic imaging, and more specifically, to photoacoustic imaging techniques that implement ergodic relays.

### BACKGROUND

Photoacoustic imaging is based on the photoacoustic effect where excitation energy is delivered to a sample and some of the delivered energy is absorbed and converted into heat, leading to transient thermoelastic expansion generating ultrasonic emissions. The ultrasonic emissions can be detected by ultrasonic transducers and the photoacoustic signals can be analyzed to construct photoacoustic images. A photoacoustic image depends on the optical absorption properties of the sample being imaged. As a consequence, it offers greater molecular specificity than conventional ultrasound imaging with the ability to detect hemoglobin, lipids, water and other light-absorbing chromophores, but with greater penetration depth than pure optical imaging modalities that rely on ballistic photons. These attributes lend photoacoustic imaging to a wide variety of applications in clinical medicine, preclinical research and basic biology for studying cancer, cardiovascular disease, abnormalities of the microcirculation and other conditions.

Background and contextual descriptions contained herein are provided solely for the purpose of generally presenting the context of the disclosure. Much of this disclosure presents work of the inventor, and simply because such work is described in the background section or presented as context elsewhere herein does not mean that such work is admitted prior art.

### SUMMARY

Certain techniques disclosed herein may be practiced with a processor-implemented method, a system comprising one or more processors and one or more processor-readable media, and/or one or more non-transitory processor-readable media.

Certain embodiments pertain to photoacoustic imaging methods. In some cases, a photoacoustic imaging method includes delivering, using at least one energy source, excitation energy into an object, the excitation energy inducing photoacoustic waves travelling through an ergodic relay in acoustic communication with the object via an acoustic medium. The photoacoustic imaging method also includes detecting, using an ultrasonic array, photoacoustic signals received through an output surface of the ergodic relay. The photoacoustic imaging method also includes time-reversing the photoacoustic signals detected by the ultrasonic array to generate a set of time-reversed photoacoustic signals and emitting, using the ultrasonic array, the set of time-reversed photoacoustic signals into the ergodic relay through the output surface. In addition, the photoacoustic imaging method includes optically imaging a surface of the acoustic medium while the ultrasonic array emits the set of time-reversed photoacoustic signals to generate a surface profile of the acoustic medium and reconstructing a two-dimensional (2D) image of the object from the surface profile. In one example, the photoacoustic imaging method also includes scanning the surface of the acoustic medium to one or more additional heights, generating one or more additional surface profiles of the acoustic medium at the one or more additional heights, reconstructing one or more additional 2D images from the one or more additional surface profiles, and combining the 2D image with the one or more additional 2D images to generate a three-dimensional (3D) image of the object.

Certain embodiments pertain to photoacoustic imaging systems. In some cases, a photoacoustic imaging system includes an ergodic relay acoustically coupled to an acoustic medium and an ultrasonic array acoustically coupled to the ergodic relay. The ultrasonic array is configured to detect photoacoustic signals received from the ergodic relay and emit time-reversed photoacoustic signals back into the ergodic relay. The photoacoustic imaging system also includes an optical imager configured to detect one or more surface profiles of the acoustic medium to reconstruct one or more two-dimensional (2D) images of an object being imaged during operation. In one aspect, the optical imager is configured to optically image a plurality of surface profiles of a surface of the acoustic medium at different heights to reconstruct a plurality of two-dimensional (2D) images of the object and combine the plurality of 2D images to construct a 3D image of the object.

Certain embodiments pertain to non-transitory machine-readable medium. In some cases, the non-transitory machine-readable medium includes instructions that, when executed by one or more processors, are configured to cause the one or more processors to perform operations. The operations include causing transmission of excitation energy into an object and recording photoacoustic signals detected by the ultrasonic array through an output surface of the ergodic relay. The operations also include time-reversing the photoacoustic signals detected by an ultrasonic array to generate a set of time-reversed photoacoustic signals and causing, using the ultrasonic array, emission of the set of time-reversed photoacoustic signals into the ergodic relay through the output surface. In addition, the operations include causing an optical imager to image a surface of the acoustic medium while the ultrasonic array emits the set of time-reversed photoacoustic signals to generate a surface profile of the acoustic medium and reconstructing a two-dimensional (2D) image of the object from the surface profile.

These and other features and embodiments will be described in more detail with reference to the drawings.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

FIG. 1A is an illustration depicting an example of components of a photoacoustic imaging system during a detection procedure, according to various embodiments.

FIG. 1B is an illustration of an example of components of the photoacoustic imaging system in FIG. 1A during an emission procedure, according to various embodiments.

FIG. 2A is an illustration depicting an example of components of a photoacoustic imaging system during a detection procedure, according to various embodiments.

FIG. 2B is an illustration of an example of components of the photoacoustic imaging system in FIG. 2A during an emission procedure, according to various embodiments.

FIG. 3 is a schematic illustration depicting an example of components of a photoacoustic imaging system during a detection procedure, according to various embodiments.

FIG. 4 is a schematic illustration of the photoacoustic imaging system in FIG. 3 during an emission procedure.

FIG. 5 is an isometric drawing depicting an example of an ergodic relay and an ultrasonic array acoustically coupled to the ergodic relay, according to an implementation.

FIG. 6 is an isometric drawing depicting an example of an ergodic relay and an ultrasonic array acoustically coupled to the ergodic relay, according to an implementation.

FIG. 7 is an isometric drawing depicting an example of an ergodic relay and an ultrasonic array acoustically coupled to the ergodic relay, according to an implementation.

FIG. 8 is an isometric drawing depicting an example of an ergodic relay and an ultrasonic array acoustically coupled to the ergodic relay, according to an implementation.

FIG. 9 is a flow diagram depicting a photoacoustic imaging method, according to some embodiments.

FIG. 10 depicts a block diagram of an example of a computing device, according to various embodiments.

FIG. 11 depicts a block diagram of an example of a computing device, according to various embodiments.

The figures and components therein may not be drawn to scale.

### DETAILED DESCRIPTION

Different aspects are described below with reference to the accompanying drawings. In the following description, numerous specific details are set forth in order to provide a thorough understanding of the presented embodiments. The disclosed embodiments may be practiced without one or more of these specific details. In other instances, well-known operations have not been described in detail to avoid unnecessarily obscuring the disclosed embodiments. While the disclosed embodiments will be described in conjunction with the specific embodiments, it will be understood that it is not intended to limit the disclosed embodiments.

### I. Introduction

In an ergodic relay, each point on an input surface has a unique time-of-arrival signature at the output surface. This time delay characteristic of the ergodic relay enables the linear summation of received photoacoustic signals at the output surface to be unmixed later, allowing the photoacoustic signal from each input point to be retrieved. Based on the time delay characteristic, a single-element transducer can function as multiple virtual transducers across the input surface. The unique signatures for each point can be used to reconstruct 3D images. However, conventional techniques require a point-by-point calibration across the ergodic relay surface or outside the ergodic relay, which can be time-consuming and change over time. Moreover, conventional techniques require computationally intensive 3D image reconstruction, which can be time-consuming as well. For instance, one such conventional technique involves single-shot photoacoustic computed tomography (PACT) where 3D single-shot imaging was performed by implementing a single-element ultrasonic transducer. An example of this technique is described in Zhang, Y., Hu, P., Li, L., Cao, R., Khadria, A., Maslov, K., Tong, X., Zeng, Y., Jiang, L., Zhou, Q., et al., “Ultrafast longitudinal imaging of haemodynamics via single-shot volumetric photoacoustic tomography with a single-element detector,” Nature Biomedical Engineering 8, pages 712-725 (2024). This conventional technique requires point-by-point calibration of the ergodic relay input surface.

Certain photoacoustic imaging techniques disclosed herein obviate the need for calibration and computationally intensive 3D image reconstruction. In some embodiments, photoacoustic imaging techniques use an energy source to deliver a single shot (e.g., single pulse laser shot) of excitation energy to an object which is converted into heat generating an initial distribution of photoacoustic signals. The initial photoacoustic signals are received at an input surface of an ergodic relay that is in acoustic communication with the object via an acoustic medium. The acoustic waves travel through the ergodic relay to an output surface. An ultrasonic array (a single ultrasonic transducer (0D array), a 1D array, or a 2D array) acoustically coupled to the output surface can detect photoacoustic signals (encoded with time delays from ergodic relay) and these detected photoacoustic signals can be recorded. The recorded photoacoustic signals can be time-reversed. In some cases, the recorded photoacoustic signals may also be amplified. The ultrasonic array can emit the time-reversed photoacoustic signals back into the ergodic relay at the output surface. The time-reversed photoacoustic signals are transformed by the ergodic relay into the initial photoacoustic signals at the input surface which are emitted into an acoustic medium (e.g., water). When the acoustic waves hit the surface of the acoustic medium, the acoustic waves are reflected downward due to the high acoustic impedance mismatch at the interface between the air and the acoustic medium. A momentum change from the reflection of acoustic waves at the medium surface causes an acoustic radiation pressure pushing the surface upward inducing a surface displacement. As the time-reversed photoacoustic signals are emitted from the ultrasonic array into the ergodic relay, the acoustic radiation pressure from the reflections of the waves causes surface displacements that form a surface profile. The surface profile carries an imprint of the initial photoacoustic signal distribution from the object. An optical imager may be used to monitor the water surface profile caused by the acoustic radiation pressure from the initial photoacoustic signals. The optical imager may acquire image frames at a frame rate synchronized with the ultrasound pulses of the time-reversed photoacoustic signals emitted by the ultrasonic array. The image frames can be used to determine surface displacements defining the surface profile. The surface displacements along the surface profile can be mapped to x-y locations to construct a 2D image of the object. In some cases, the surface of the acoustic medium is scanned to multiple heights and a 2D image is generated at each height or a subset of the heights. The 2D images at different heights can be combined to form a 3D image of the object.

### Calibration-Free Hardware Reconstruction

One advantage to the photoacoustic imaging methods disclosed herein is that calibration of the system is unnecessary because an image is formed using a physical time reversal of the received acoustic signals. The ergodic process is time-reversed, allowing the system to reconstruct the image based on the captured signals naturally. These methods enhance the efficiency of the imaging system in both calibration and computation.

### Water Surface or Other Acoustic Medium Surface

In some embodiments, the photoacoustic imaging techniques aim to detect the surface profile of the water or other acoustic medium as the photoacoustic signals are time-reversed from the physical ultrasonic transducer array, passed back through the ergodic relay, and returned to the acoustic medium. The surface of the acoustic medium (e.g., water surface) changes height due to acoustic radiation pressure, and profilometry can be employed to detect these changes. Some examples of profilometry techniques that can be used are described in Van der Jeught, S. and Dirckx, J. J., “Real-time structured light profilometry: a review, Optics and Lasers in Engineering 87, 18-31 (2016).

By mapping the changes in height of the medium surface, a 2D image can be formed at a time. This technique uses the surface of the acoustic medium as a flexible membrane that responds to the acoustic field, making the ultrasound pattern visible. In an alternative implementation, a physical membrane may also be used above the acoustic medium for greater stability. Some examples of physical membrane that can be used include Mylar film, polydimethylsiloxane sheets, and polyurethane membranes.

### 3D Imaging

In some embodiments, the photoacoustic imaging techniques image the surface of the acoustic medium (medium surface) at a high frame rate to form a 2D image at a time. In one example, a high frame rate may be in a range between 1 KHz and 10 KHz, depending on the ultrasound propagation distance. The 2D image acquisition by the optical imager is timed with the ultrasound pulses emitted by the ultrasonic array. The 3D image can be formed quickly by scanning the height of the medium surface while repeatedly acquiring 2D images. For example, a 3D image may be formed in 10, 100, 1000 Hz, depending on the number of slices to be included.

### II. Photoacoustic Imaging Systems

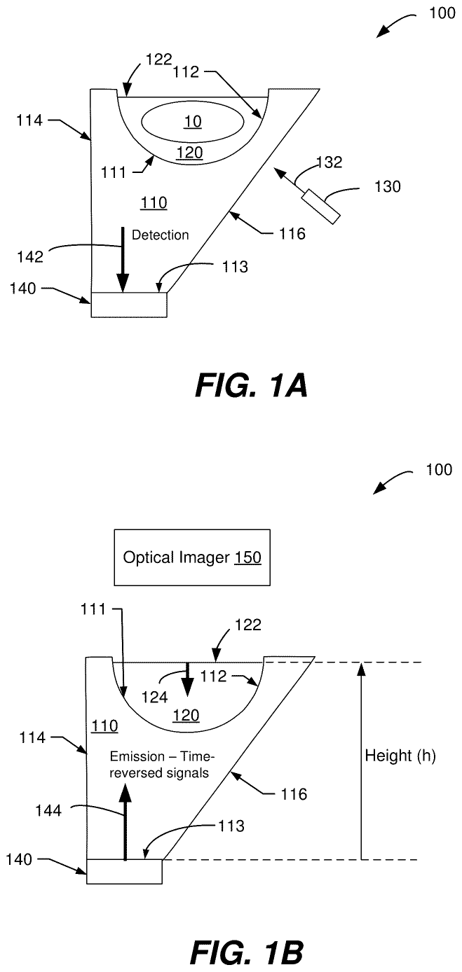

In some embodiments, photoacoustic imaging systems employ an ultrasonic array (e.g., 0D array (single transducer element), 1D array, or 2D array) coupled to an ergodic relay to record photoacoustic signals that can be used in single-shot 3D imaging. An example of such a photoacoustic imaging system is shown in and described below with reference to FIGS. 1A and 1B. In these embodiments, only one shot (e.g., a single pulsed laser shot) of excitation energy may be required to obtain a data set needed to generate a 3D image of the object. The recorded photoacoustic signals are time reversed and emitted by the ultrasonic array back into the ergodic relay. The ergodic array physically time-reverses the signals received by the ultrasonic array which may obviate the need for computer-based image reconstruction. This hardware-based image reconstruction technique is extremely fast and avoids the need for point-by-point calibration. This technique may be limited by the speed of sound, as it depends on the sound propagation time through the ergodic relay and the rest of the imaging system.

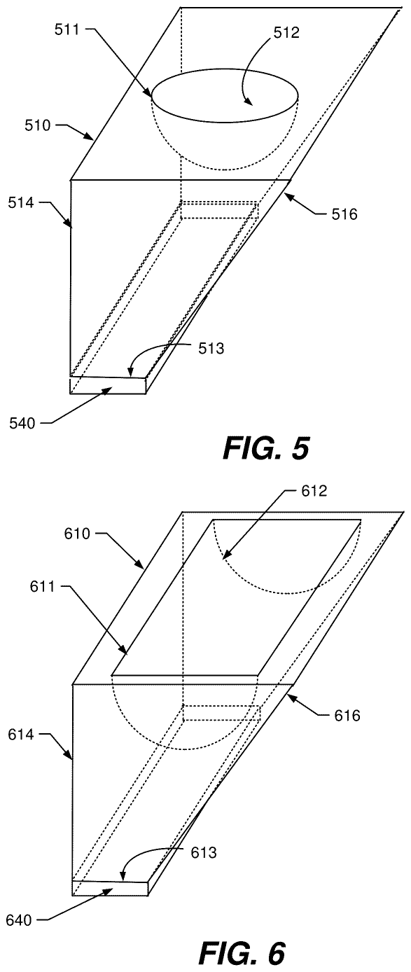

FIG. 1A is an illustration depicting an example of components of a photoacoustic imaging system 100 during a detection procedure, according to various embodiments. The photoacoustic imaging system 100 includes an ergodic relay 110 made of a solid low-loss acoustic propagation medium material (e.g., silica glass). The illustration includes a cross-section of the ergodic relay 110. The ergodic relay 110 includes a receptacle 111 having a hemispherical input surface 112. The ergodic relay 110 also includes a first side surface 114, a second side surface 116, and an output surface 113. In other implementations, other shapes and material combinations may be used. Some examples of shapes of ergodic relays are illustrated in, and described with reference to, FIGS. 5-8.

During the detection procedure, the receptacle 111 is at least partially filled with an acoustic medium (e.g., water, acoustic gel, etc.) 120. In the illustrated example, the receptacle 111 is shown as an integral part of the ergodic relay 110. In other examples, the receptacle 111 may be a separate component acoustically coupled to the ergodic relay 110. During the detection procedure, at least a portion of an object being imaged may be submerged in or surrounded by an acoustic medium located between the ergodic relay and the object. In the illustrated example, the object 10 is shown submerged in the acoustic medium 120.

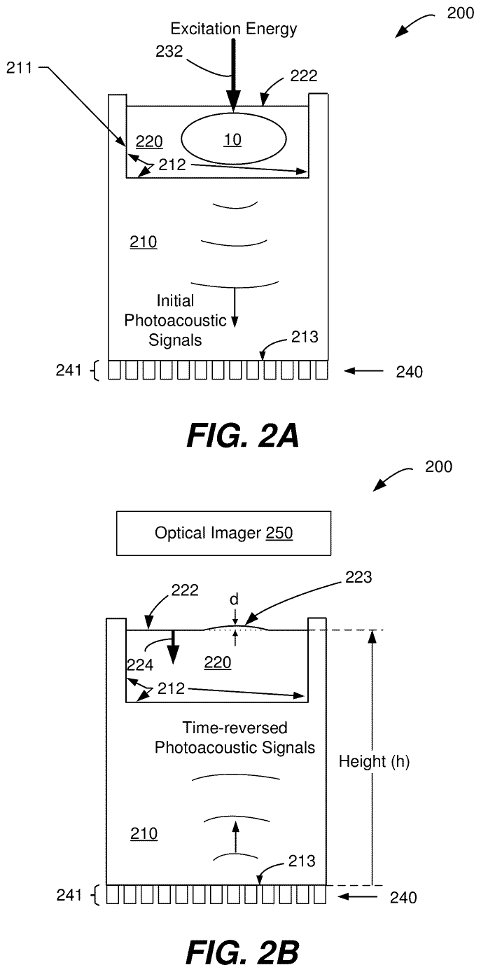

The photoacoustic imaging system 100 also includes an energy source system 130 with one or more energy sources that can deliver excitation energy 132 (e.g., a single laser shot from a pulsed laser) to the object 10 to induce an initial distribution of photoacoustic signals. The energy source(s) and any other components of the energy source system 130 are positioned and located to deliver the excitation energy 132 to the object 10. In FIG. 1A, the energy source system 130 is positioned to the outside of the second side surface 116 of the ergodic relay 110 to deliver excitation energy 132 from the periphery through the optically transparent ergodic relay 110 into the object 10. When the excitation energy 132 is light, this configuration may provide dark field illumination that can mitigate the issue of overwhelming surface signals. By delivering light from the periphery rather than directly over the central area, the amount of light interacting with superficial structures may be reduced. In other examples, the energy source system 130 may be in other locations such as above the object 10 and/or the excitation energy 132 may be delivered from other locations (e.g., excitation energy 232 in FIG. 2A is delivered from above the object 10). For example, when the object 10 being imaged is a human breast where a patient is lying over the ergodic relay, the energy source system may deliver the excitation energy from the periphery. In another example, such as when imaging a small animal, the energy source system may deliver the excitation energy from above the object 10.

The photoacoustic imaging system 100 also includes an ultrasonic array 140 acoustically coupled to the output surface 113 of the ergodic relay 110. In the illustrated example, the ultrasonic array 140 is a 2D ultrasonic array. In other implementations, a single element ultrasonic transducer (0D array) or 1D linear ultrasonic array may be used. During the detection procedure, the ultrasonic array 140 can detect photoacoustic signals (detection represented by arrow 142) traveling from the ergodic relay 110 through the output surface 113. The detected photoacoustic signals are encoded with time delays introduced by the ergodic relay 110. The ultrasonic array 140 may be acoustically coupled directly to the ergodic relay 110 or via an acoustic coupling material (e.g., polyester resin) to the output surface 113.

FIG. 1B is an illustration of an example of components of the photoacoustic imaging system 100 in FIG. 1A during an emission procedure, according to various embodiments. The illustration includes a cross-sectional representation of the ergodic relay 110. During the emission procedure, the energy source system 130 is not employed and may be omitted as shown. Also, during the emission procedure, the object 10 is not located in the acoustic medium 120. In FIG. 1B, the photoacoustic imaging system 100 includes an optical imager 150. In some implementations, the optical imager 150 includes one or more light detectors (e.g., image sensors). In some cases, the optical imager 150 may include an optical system with additional optical elements (e.g., one or more mirrors, one or more lenses, etc.).

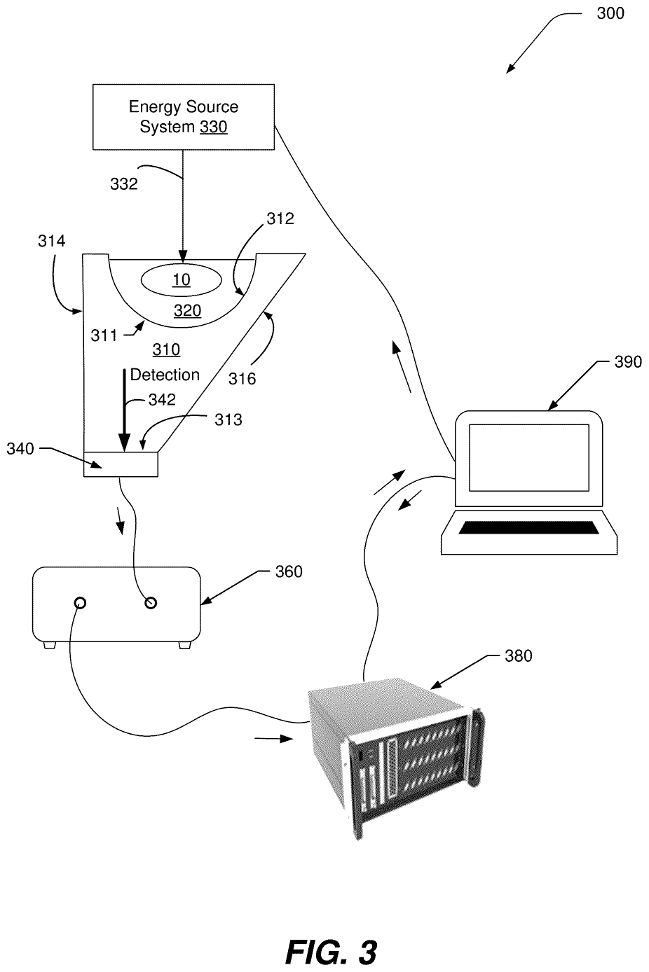

During the emission procedure, the ultrasonic array 140 or a computing device (e.g., computing device 390 in FIG. 3) in communication with the ultrasonic array 140 may be used to time reverse the photoacoustic signals detected by the ultrasonic array 140. The ultrasonic array 140 can emit the time-reversed photoacoustic signals (emission represented by arrow 144) at the output surface 113 into the ergodic relay 110. In travelling between the output surface 113 and the input surface 112, the time-reversed photoacoustic signals are transformed back into the initial distribution of photoacoustic signals. As the time-reversed photoacoustic signals are emitted from the ultrasonic array 140 into the ergodic relay 110, the acoustic radiation pressure from the reflection of the acoustic waves at the medium surface 122 causes surface displacements, dₙ, that in combination form a surface profile that carries an imprint of the initial photoacoustic signal distribution from the object 10. The optical imager 150 acquires image frames at a frame rate that is synchronized with the timing of the emissions of the ultrasonic array 140. The photoacoustic imaging system 100 can use the image frames to determine the surface displacements. In some cases, profilometry may be used to detect the surface profile. The surface displacements along the surface profile can be mapped to x-y coordinates to construct a 2D image of the object 10. In FIGS. 1A and 1B, the surface 222 of medium 220 is shown located (prior to displacements) at a plane at a height, h, relative to a reference plane at the output surface 113 of the ergodic relay 110. The surface 122 of the acoustic medium 120 is scanned (denoted by arrow 124) over multiple heights and a 2D image is generated at each height or a subset of the heights. The 2D images acquired at different heights can be combined to generate a 3D image of the object 10.

FIG. 2A is an illustration depicting an example of components of a photoacoustic imaging system 200 during a detection procedure, according to various embodiments. Some of the elements shown in FIGS. 2A and 2B are similar or analogous to elements shown in FIGS. 1A and 1B. For the sake of brevity, the prior discussion of such similar or analogous elements with regard to FIGS. 1A and 1B may be assumed to be equally applicable, unless indicated otherwise in the following discussion, to the similar or analogous counterparts of those elements in FIGS. 2A and 2B that share the same last two digits in their respective callouts as in FIGS. 1A and 1B.

The photoacoustic imaging system 200 includes an ergodic relay 210 made of a solid low-loss acoustic propagation medium material. The illustration includes a cross-sectional representation of the ergodic relay 110. As shown, the ergodic relay 210 includes an integral receptacle 211 having an input surface 212. The ergodic relay 210 also includes a first side surface 214, a second side surface 216, and an output surface 213. In other implementations, other shapes and material combinations may be used. Some examples of different shapes of ergodic relays are illustrated in, and described with reference to, FIGS. 5-8.

The photoacoustic imaging system 200 also includes an ultrasonic array 240 acoustically coupled (directly or via an acoustic coupling material) to the output surface 213. In the illustrated example, the ultrasonic array 240 is a 2D ultrasonic array including a 13×13 matrix of transducer elements 241. In other implementations, other 2D ultrasonic arrays, a single element ultrasonic transducer (0D array), or 1D (linear) ultrasonic array may be used.

During the detection procedure, the receptacle 211 is at least partially filled with an acoustic medium 220 and the object 10 being imaged is submerged in the acoustic medium 220. An excitation energy 232 (e.g., single laser shot) is delivered to the object 10 from above. In another implementation, the energy source system 130 may be configured to deliver the excitation energy 232 from the periphery. The excitation energy 232 induces photoacoustic waves that are received at the input surface 212 of the ergodic relay 210. During the detection procedure, the ultrasonic array 240 can detect photoacoustic signals at the output surface 213.

FIG. 2B is an illustration of an example of components of the photoacoustic imaging system 200 in FIG. 2A during an emission procedure, according to various embodiments. The illustration includes a cross-sectional representation of the ergodic relay 110. During the emission procedure, the object 10 is not located in the acoustic medium 220. During the emission procedure, the photoacoustic imaging system 200 includes an optical imager 250. The ultrasonic array 240 or a computing device (e.g., computing device 390 in FIG. 3) in communication with the ultrasonic array 240 may be used to time reverse the photoacoustic signals detected by the ultrasonic array 240. The ultrasonic array 240 can emit the time-reversed photoacoustic signals at the output surface 213 into the ergodic relay 210. In travelling between the output surface 213 and the input surface 212, the time-reversed photoacoustic signals are transformed back into the initial distribution of photoacoustic signals. The acoustic radiation pressure from the reflection of the acoustic waves at the surface 222 of the acoustic medium 220 causes surface displacements d_{i=1toN}, that in combination form a surface profile that carries an imprint of the initial photoacoustic signal distribution from the object 10. In FIG. 2B, an example of an upward surface displacement, d, 223 is shown at medium surface 222 at a frame acquisition time. The optical imager 250 acquires images at a frame rate that is synchronized with the timing of the emissions of the ultrasonic array 240. The photoacoustic imaging system 200 can form a surface profile using the surface displacements determined from the image frames. The surface displacements along the surface profile can be mapped to x-y coordinates to construct a 2D image of the object 10. The surface 222 of the acoustic medium 220 is scanned (denoted by arrow 224) over multiple heights and a 2D image is generated at each height or a subset of the heights. The 2D images acquired at different heights can be combined to generate a 3D image of the object 10.

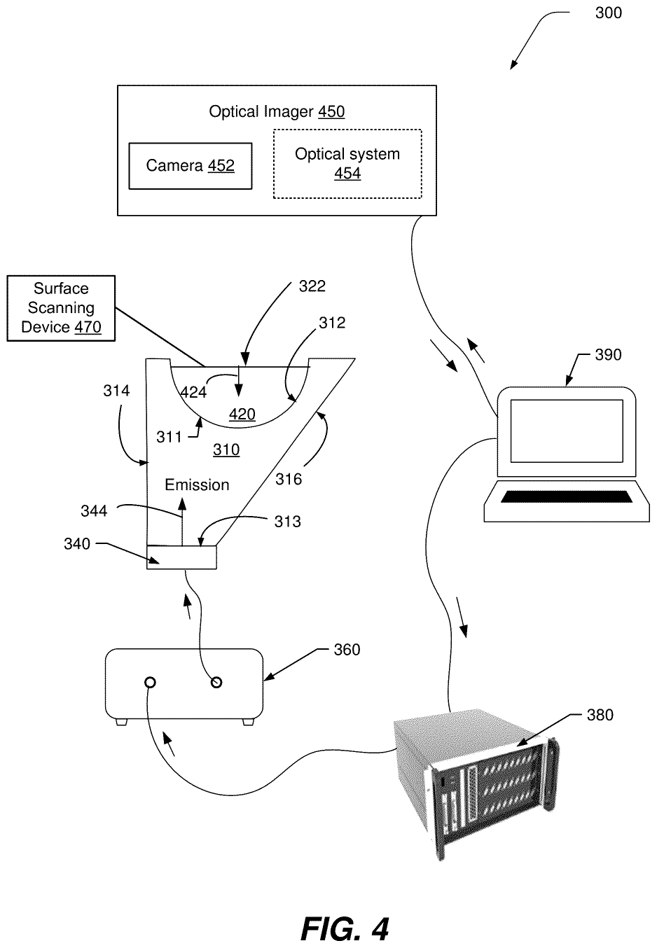

FIG. 3 is a schematic illustration depicting an example of components of a photoacoustic imaging system 300 during a detection procedure, according to various embodiments. FIG. 4 is a schematic illustration of the photoacoustic imaging system 300 in FIG. 3 during an emission procedure. Some of the elements shown in FIGS. 3 and 4 are similar or analogous to elements shown in FIGS. 1A and 1B. For the sake of brevity, the prior discussion of such similar or analogous elements with regard to FIGS. 1A and 1B may be assumed to be equally applicable, unless indicated otherwise in the following discussion, to the similar or analogous counterparts of those elements in FIGS. 3 and 4 that share the same last two digits in their respective callouts as in FIGS. 1A and 1B.

The photoacoustic imaging system 300 includes an ergodic relay 310 made of a solid low-loss acoustic propagation medium material. The illustration includes a cross-sectional representation of the ergodic relay 310. The ergodic relay 310 includes an integral receptacle 311 having a hemispherical input surface 312. In other examples, the receptacle 311 may be a separate component. The ergodic relay 310 also includes a first side surface 314, a second side surface 316, and an output surface 313. In other implementations, other shapes and material combinations may be used. Some examples of shapes of ergodic relays are illustrated in and described with reference to FIGS. 5-8. During the detection procedure of this implementation, the receptacle 311 is at least partially filled with an acoustic medium 420 and the object 10 being imaged is submerged in the acoustic medium 420.

The photoacoustic imaging system 300 also includes an energy source system 330 with energy source(s) that can deliver an excitation energy 332 to the object 10. The excitation energy 332 can induce an initial distribution of photoacoustic signals emitted from the object 10 due to the photoacoustic effect. The energy source(s) and any other components of the energy source system 330 are configured to deliver excitation energy 332 to the object 10. In the illustrated example, the energy source system 330 is configured to deliver excitation energy 332 from above the object 10. As another implementation, the energy source system 330 may be configured to deliver excitation energy 332 from the periphery through an optically transparent ergodic relay 310 as illustrated in FIG. 1A.

According to various embodiments, the photoacoustic imaging system may include an energy source system with one or more energy sources that can provide excitation energy. Some examples of types of energy that can be used include laser energy, RF/microwave, x-ray, gamma ray, protons, and electrons. In some cases, at least one of the energy sources is a pulsed laser. For example, at least one of the energy sources may be a pulsed laser that can generate near infrared pulses having a wavelength or narrow band of wavelengths in a range from about 700 nm to about 1000 nm. As another example, at least one of the energy sources may be a pulsed laser that can generate near infrared pulses having a wavelength or narrow band of wavelengths in a range from about 600 nm to about 1100 nm. In yet another example, at least one of the energy sources may be a pulsed laser that can generate near infrared pulses with a wavelength or narrow band of wavelengths greater than 760 nm. In yet another example, at least one of the energy sources may be a pulsed laser that can generate near infrared pulses with a wavelength or narrow band of wavelengths greater than 1000 nm. In another example, at least one of the energy sources may be a pulsed laser that can generate a 1064-nm laser beam. A commercially-available example of a suitable pulsed laser is the PRO-350-10, Quanta-Ray® laser with a 10-Hz pulse repetition rate and 8 ns-12 ns pulse width sold by Spectra-Physics®. The low optical attenuation of 1064 nm light or other near infrared light can be used to deeply penetrate to, e.g., a depth of 4 cm, into biological tissues. In one aspect, at least one of the energy sources is a tunable narrow-band pulsed laser such as, e.g., one of a quantum cascade laser, an interband cascade laser, an optical parametric oscillator, or other pulsed laser that can be tuned to different narrow bands (e.g., a near-infrared band). In implementations that include a energy source in the form of a pulsed laser, the pulse repetition rate may be about 10-Hz in some cases, about 20-Hz in other cases, about 50-Hz in other cases, and about 100-Hz in other cases. In another case, the pulse repetition rate is in a range from about 10-Hz to about 100-Hz. Alternatively, at least one of the energy sources may be a continuous wave laser source that is chopped, modulated and/or gated.

Returning to FIG. 3, the photoacoustic imaging system 300 also includes an ultrasonic array 340 acoustically coupled to the output surface 313 of the ergodic relay 310. In the illustrated example, the ultrasonic array 340 is a 2D ultrasonic array. In other implementations, a single element ultrasonic transducer (0D array) or 1D linear ultrasonic array may be used. During the detection procedure, the ultrasonic array 340 can detect photoacoustic signals (detection represented by arrow 342) traveling from the ergodic relay 310 through the output surface 313. The ultrasonic array 340 may be acoustically coupled directly to the ergodic relay 310 or via an acoustic coupling material (e.g., polyester resin) to the output surface 313.

The photoacoustic imaging system 300 also includes amplifier(s) 360 in electronic communication with the ultrasonic array 340 to receive the detected photoacoustic signals. The amplifier(s) 360 can boost (increasing amplitude) the photoacoustic signals transmitted from the ultrasonic transducer array 440 and/or transmitted to ultrasonic transducer array 440. In some cases, the amplifier gain may be based on one or more of a minimum signal-to-noise ratio and one or more operating parameters of the optical imager 350 or other components of the photoacoustic system 300. In certain aspects, the gain of may be at least about 5 dB, at least about 7 dB, at least about 9 dB, at least about 11 dB, at least about 13 dB, at least about 15 dB, at least about 17 dB, at least about 19 dB, at least about 21 dB, at least about 23 dB, at least about 25 dB, or at least about 30 dB. In one case, the gain is about 20 dB.

The photoacoustic imaging system 300 also includes one or more data acquisition systems (DAQs) 380 in electronic communication with the amplifier(s) 360 to receive the boosted photoacoustic signals and a computing device 390 in electronic communication with the DAQs 380 to receive the digitized photoacoustic data and in electronic communication with the energy source system 330 to transmit control signals. The DAQ(s) 380 may record the photoacoustic signals at time intervals defined by a sampling frequency (e.g., in a range from about 4 MHz to about 100-Hz). The computing device 390 may execute instructions to perform functions of a photoacoustic imaging method. The computing device 390 executes instructions to perform functions of the photoacoustic imaging method. For example, the computing device 390 generates time-reversed waveforms from the digitized, amplified photoacoustic data acquired by the DAQ 380 and sends the time-reversed data to a signal-generation module, such as an arbitrary waveform generator or an ultrasound transmit beamformer. The signal-generation module converts the data to analog time-reversed drive signals and delivers those signals, directly or through another amplifier, to the ultrasonic array 340.

During the emission procedure, the energy source system 330 is not employed and may be omitted as shown in FIG. 4. Also, during the emission procedure, the object 10 is not located in the acoustic medium 320. In FIG. 4, the photoacoustic imaging system 300 includes an optical imager 450 in optical communication with the surface 322 of the acoustic medium 320. The optical imager 450 includes a camera 452 with one or more light detectors (e.g., image sensors) and an optional (denoted by dashed line) optical system 454. The optical system 454 may include one or more optical components (e.g., lens(es), optical filter(s), mirror(s), beam steering device(s), beam-splitter(s), optical fiber(s), relay(s), and/or beam combiner(s)) configured to deliver light reflected from the surface 422 of the acoustic medium 420 to the camera 452. In some cases, the optical imager 450 may also include an illumination source that can provide illumination delivered to the surface 322. In some cases, the optical imager 450 may also include a spatial light modulator. The computing device 390 is in electronic communication with the optical imager 450 to transmit control signals to synchronize frame acquisition of the camera 452 with emission of the time-reversed photoacoustic signals from the ultrasonic array 340 and to receive image data from the optical imager 450.

The photoacoustic imaging system 300 also includes a surface scanning device 470 that is in communication with the surface 322 of the acoustic medium 420. The surface scanning device 470 can scan the surface 322 to different heights. An example of a surface scanning device is a water pump.

The ultrasonic array 240 or computing device 390 can time reverse the photoacoustic signals detected during the detection procedure illustrated in FIG. 3. The ultrasonic array 240 can emit the time-reversed photoacoustic signals at the output surface 313 into the ergodic relay 210. In travelling between the output surface 313 and the input surface 312, the time-reversed photoacoustic signals are transformed back into the initial distribution of photoacoustic signals.

The camera 452 of the optical imager 250 is configured to acquire images at a frame rate that is synchronized with the timing of the emissions of the ultrasonic array 240. The photoacoustic imaging system 200 can form a surface profile using the surface displacements determined from the image frames. The surface displacements along the surface profile can be mapped to x-y coordinates to construct a 2D image of the object 10. During the emission procedure, the surface scanning device 470 can scan the surface 312 of the acoustic medium 220 is scanned (denoted by arrow 424) to multiple heights. A 2D image may be generated at each height or a subset of the heights. 2D images acquired at different heights can be combined to generate a 3D image of the object 10.

### A. Ergodic Relay

As used herein, an ergodic relay generally refers to a lossless or low-loss acoustic propagation medium that scrambles input acoustic waves inside the medium by internally reflecting the waves at boundaries which results in distinct time delay characteristics at an output location for each input location. The waves are reflected at the boundaries due to the discontinuity in acoustic transmissivity between the medium of the ergodic relay and the air or other medium outside the boundaries. If the ergodic relay is considered lossless and the boundaries perfect reflectors, an acoustic wave at a particular input location propagates to an output location along a unique path relative to the paths of other acoustic waves at other input locations.

In accordance with some embodiments, the ergodic relay may be made of different low-loss or lossless acoustic propagation material or materials. In some cases, the ergodic relay is made of a solid material such as a fused silica. In some cases, an ergodic relay may be made of a combination of materials such as a glass container filled with an acoustic medium such as water.

### Expansion of View

An ergodic array includes an input surface and an output surface. An input surface of an ergodic relay generally refers to a surface that is in acoustic communication (e.g., via an acoustic medium) with an object being imaged to receive photoacoustic signals. An output surface of an ergodic relay generally refers to a surface that is acoustically coupled (e.g., directly or via an acoustic coupling material such as a polyester resin) to the ultrasonic array to enable receiving photoacoustic signals from the ergodic relay.

In some cases, an ergodic relay includes a planar input surface. In other cases, an ergodic relay includes an input surface with curvature (curved input surface) such as a hemispherical input surface (e.g., hemispherical input surface 112 in FIGS. 1A and 1B, hemispherical input surface 312 in FIGS. 3 and 4, and hemispherical input surface 512 in FIG. 5), a partial cylindrical input surface (e.g., half cylindrical input surface 532 in FIG. 6), a conical input surface, or other curved input surface. A curved input surface may advantageously expand the view range around the object being imaged as compared with a planar input surface that might have a limited view. The larger view of a curved input surface may advantageously allow for better reconstruction of the acoustic signals from different angles, leading to improved image quality and reduced artifacts.

### Improved Elevational Resolution

Conventional photoacoustic imaging systems with 1D ultrasonic arrays did not include ergodic relays and were sometimes prone to having poor spatial resolution in the elevational (Z) direction, which restricts the array's capability to capture detailed 3D images. The photoacoustic imaging systems disclosed herein include an ergodic relay which can spatially randomize the ultrasound field, allowing the ultrasonic array to capture signals from multiple angles, effectively reconstructing a 3D volumetric image while still employing the simpler, more accessible 1D ultrasonic array. These photoacoustic imaging systems may address the limitations of the conventional 1D ultrasound array systems.

### Examples of Shapes of Ergodic Relays

Different shapes of ergodic relays may be used in accordance with various embodiments. Some examples of shapes of ergodic relays are shown in FIGS. 5-8.

FIG. 5 is an isometric drawing depicting an example of an ergodic relay 510 and an ultrasonic array 540 acoustically coupled to the ergodic relay 510, according to an implementation. The ergodic relay 510 is an example of an implementation of the ergodic relay 110 shown in FIGS. 1A and 1B and an example of an implementation of the ergodic relay 310 shown in FIGS. 3 and 4. The ergodic relay 510 is made of a solid low-loss acoustic propagation medium material (e.g., silica glass) and includes an integral receptacle 511 having a hemispherical input surface 512. The ergodic relay 510 also includes a first side surface 514, a second side surface 516, and an output surface 513 between the ergodic relay 610 and the ultrasonic array 640. In the illustrated example, the ultrasonic array 640 is a 2D ultrasonic array. In other implementations, a single element ultrasonic transducer (0D array) or 1D (linear) ultrasonic array may be used.

FIG. 6 is an isometric drawing depicting an example of an ergodic relay 610 and an ultrasonic array 640 acoustically coupled to the ergodic relay 610, according to an implementation. The ergodic relay 610 is made of a solid low-loss acoustic propagation medium material and includes an integral receptacle 611 having a half cylindrical input surface 612. The ergodic relay 610 also includes a first side surface 614, a second side surface 616, and an output surface 613 at the interface between the ergodic relay 610 and the ultrasonic array 640. In the illustrated example, the ultrasonic array 640 is a 2D ultrasonic array. In other implementations, a single element ultrasonic transducer (0D array) or 1D (linear) ultrasonic array may be used.

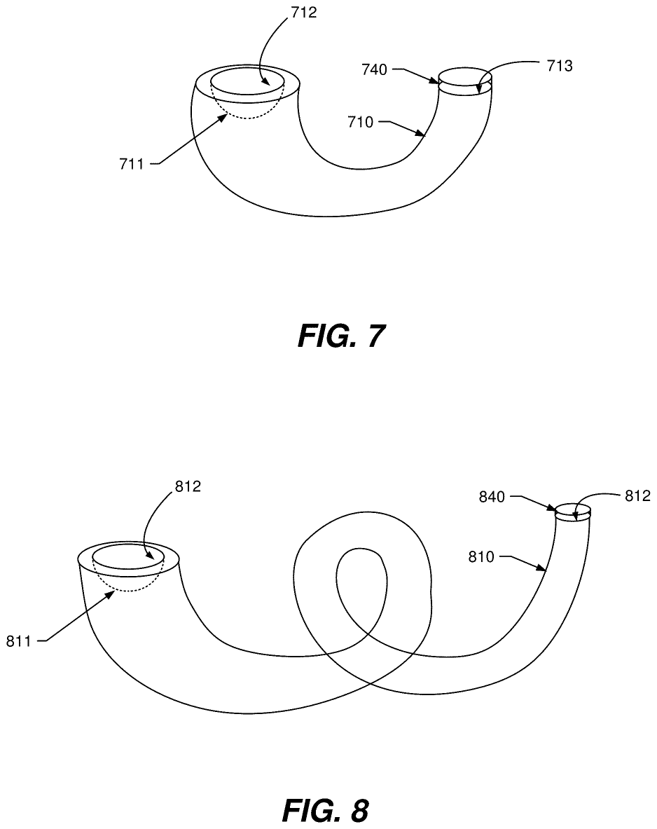

FIG. 7 is an isometric drawing depicting an example of an ergodic relay 710 and an ultrasonic array 740 acoustically coupled to the ergodic relay 710, according to an implementation. The ergodic relay 710 is made of a solid low-loss acoustic propagation medium material and includes an integral receptacle 711 having a hemispherical input surface 712. The ergodic relay 710 also includes a first side surface 714, a second side surface 716, and an output surface 713 at the interface between the ergodic relay 710 and the ultrasonic array 740. In the illustrated example, the ultrasonic array 740 is a 2D ultrasonic array. In other implementations, a single element ultrasonic transducer (0D array) or 1D (linear) ultrasonic array may be used.

FIG. 8 is an isometric drawing depicting an example of an ergodic relay 810 and an ultrasonic array 840 acoustically coupled to the ergodic relay 810, according to an implementation. The ergodic relay 810 is made of a solid low-loss acoustic propagation medium material and includes an integral receptacle 811 having a hemispherical input surface 812. The ergodic relay 810 also includes a first side surface 814, a second side surface 816, and an output surface 813 at the interface between the ergodic relay 810 and the ultrasonic array 840. In the illustrated example, the ultrasonic array 840 is a 2D ultrasonic array. In other implementations, a single element ultrasonic transducer (0D array) or 1D (linear) ultrasonic array may be used.

In some embodiments, the input surface of the ergodic relay does not have a direct line of sight to the ultrasonic array to ensure that the photoacoustic signals are scrambled before being detected by the ultrasonic array. This geometric occlusion ensures that the photoacoustic waves originating at the input surface undergo multiple internal reflections and path-length diversifications inside the relay before reaching the ultrasonic array. By enforcing such indirect propagation, the acoustic wavefronts become effectively scrambled, which promotes formation of a spatially incoherent, diffuse acoustic field at the array. This scrambling supports robust time-reversal focusing or reconstruction and minimizes potential artifacts associated with direct ballistic or near-ballistic propagation paths. For example, the input surface 712 of the ergodic relay 710 in FIG. 7 does not have a line of sight to the output surface 713 interface with the ultrasonic array 740 due to the curved horn shape of the ergodic relay 710. As another example, the input surface 812 of the ergodic relay 810 in FIG. 8 does not have a line of sight to the output surface 813 interface with the ultrasonic array 840 due to the multiple curves in the shape of the ergodic relay 810.

### B. Ultrasonic Array

In various embodiments, photoacoustic imaging systems include at least one ultrasonic array in acoustic communication with the ergodic relay. In some cases, a 2D ultrasonic array (e.g., ultrasonic array 240 in FIGS. 2A and 2B) with a two-dimensional matrix of transducer elements (e.g., transducer elements 241 in FIGS. 2A and 2B) may be implemented. In some cases, a 1D (linear) ultrasonic array may be implemented. In some cases, a 0D (single element) ultrasonic array may be implemented. In some cases, a combination of ultrasonic arrays may be implemented. Any suitable dimensions for the arrangement of transducer elements may be used according to various embodiments.

### Single-Element Ultrasonic Transducer (0D Array) vs 1D Array or 2D Array

Extending from a single-element ultrasonic transducer (0D array) to a 1D or 2D ultrasonic array allows for the reception of more energy from diverse spatial locations via the ergodic relay. This approach may enhance the signal-to-noise ratio, spatial resolution, and image fidelity, but at the expense of system complexity and cost.

Generally speaking, 1D and 2D ultrasonic arrays have greater energy collection efficiency than a single transducer element. The 1D and 2D arrays may enable imaging of smaller detailed features of an object for the equivalent data acquisition duration. For example, small blood vessels may produce weaker signals than large blood vessels in a tissue sample and the energy collected by a single transducer in a short period of time may not be sufficient to image the smaller blood vessels. To be able to image smaller blood vessels with a single transducer may require scanning single transducer element which would require a longer acquisition time to be able to collect adequate data. Whereas with implementations that employ a 1D or a 2D ultrasonic array, the independent transducer elements in the arrays each collect energy and in combination can acquire adequate data during a shorter acquisition time.

Implementing a 0D array may be advantageous to enable a system to be in a compact form such as in a wearable device, particularly if there are no time constraints for scanning the single element transducer. For example, in a neonatal intensive care unit (NICU), a photoacoustic imaging system in a compact form with a single transducer element that can be scanned overnight may be advantageous.

### Reduction of the Number of Physical Transducer Elements

Conventional 3D photoacoustic computed tomography (PACT) methods typically require either a large number of physical ultrasonic detector elements or a single-element ultrasonic transducer that must be scanned to mimic an array. While the former is expensive, the latter is time-consuming. Generating a high-quality 3D image from these conventional methods might require an order of a quarter million ultrasonic transducer elements.

The photoacoustic imaging techniques of embodiments dramatically reduce the number of physical ultrasonic transducers needed to acquire a high-quality 3D image because the ergodic relay converts spatially distributed photoacoustic fields into temporally diversified waveforms detectable by a much smaller sensing aperture. The relay produces a rich set of path-length variations, internal reflections, and mode diversifications, so that each transducer element receives a temporally encoded mixture of acoustic energy originating from many spatial locations. As a result, spatial information that would otherwise require a dense transducer array is effectively compressed into the time domain. Time-reversal or equivalent decoding algorithms then invert this encoding to reconstruct the three-dimensional distribution of photoacoustic sources. This multiplexed acquisition paradigm allows high-fidelity volumetric imaging with far fewer physical elements than conventional systems, thereby reducing hardware complexity, cost, and cabling without compromising spatial resolution or field of view.

### C. Amplifier—Signal Amplification for Time Reversal

In various embodiments, photoacoustic imaging systems include at least one amplifier (e.g., amplifier 360 in FIGS. 3 and 4) in electronic communication with the ultrasonic array to transmit amplified time reversed photoacoustic signals and/or with the computing device to receive control signals. In some cases, amplifier gain may be based on one or more of a minimum signal-to-noise ratio and one or more operating parameters of the optical imager 350 or other components of the photoacoustic system 300. In certain aspects, the gain of may be at least about 5 dB, at least about 7 dB, at least about 9 dB, at least about 11 dB, at least about 13 dB, at least about 15 dB, at least about 17 dB, at least about 19 dB, at least about 21 dB, at least about 23 dB, at least about 25 dB, or at least about 30 dB.

By amplifying the time-reversed signal before transmission into the ergodic relay, the photoacoustic imaging systems can achieve larger displacement of the acoustic medium surface. This amplified displacement enhances the accuracy of the reconstructed image by providing a more pronounced and detectable change in the surface profile.

### III. Photoacoustic Imaging Methods

### 2D and 3D Imaging

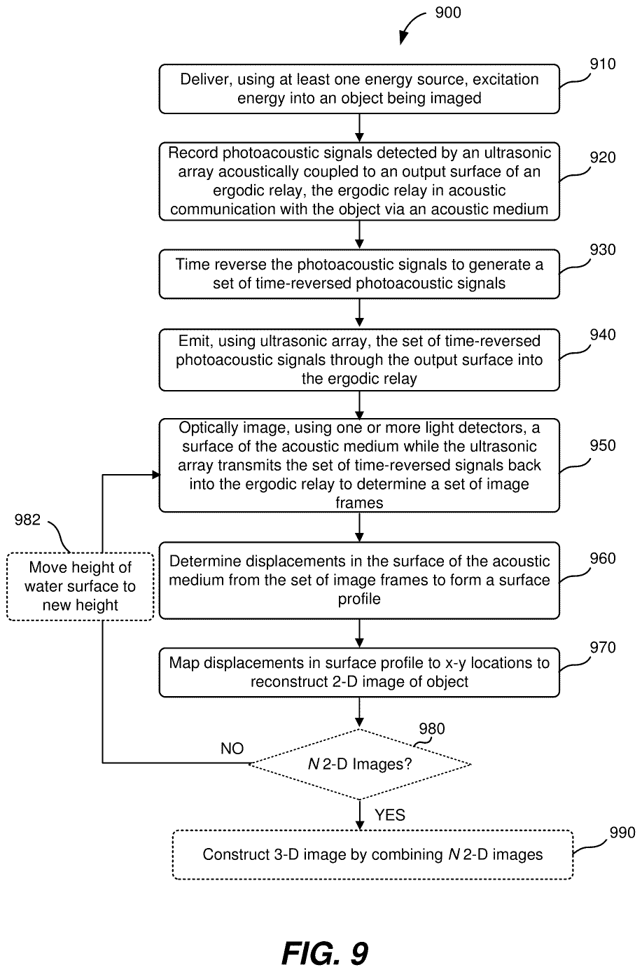

FIG. 9 is a flow diagram 900 depicting a photoacoustic imaging method, according to some embodiments. Various examples of photoacoustic imaging systems (e.g., photoacoustic imaging system 100 in FIGS. 1A and 1B, photoacoustic imaging system 200 in FIGS. 2A and 2B, and photoacoustic imaging system 100 in FIGS. 1A and 1B) with ergodic relays described herein can be used to implement the operations. Blocks of the flow diagram 900 may be executed by one or more processors of one or more computing devices. Some examples of computing devices are shown in and described herein in connection with FIGS. 3 and 4 and FIG. 12. Note that, in some embodiments, at least one of the one or more processors may be disposed in the photoacoustic imaging system. Alternatively, data obtained from the photoacoustic imaging system may be transmitted from the computing device to a second computing device remote from or separate from the photoacoustic imaging system. In some implementations, the blocks of flow diagram 900 may be executed in an order other than what is shown in FIG. 9. In some embodiments, one or more blocks of process 900 may be omitted, and/or two or more blocks may be executed substantially in parallel.

At 910, the photoacoustic imaging method can begin by causing at least one energy source to deliver an excitation energy into an object being imaged. In some cases, the at least one energy source includes a laser such as a pulsed laser. In these cases, the excitation energy may be in the form of a single pulse laser shot. In other implementations, other forms of excitation energy may be used such as RF/microwave, x-ray, gamma ray, protons, and electrons. The excitation energy is converted into heat generating an initial distribution of photoacoustic signals. The initial photoacoustic signals are received at an input surface of an ergodic relay where the input surface is in acoustic communication via an acoustic medium with the object being imaged. In various implementations, the input surface is an internal curved surface (e.g., hemispherical input surface, partial cylindrical input surface, etc.). The ergodic relay scrambles acoustic waves by internally reflecting the waves at its boundaries. Acoustic waves travel through the ergodic relay to an output surface at an acoustically coupled interface with an ultrasonic array.

In certain implementations, the excitation energy is light delivered from a periphery of the ergodic relay. An illustrated example of excitation energy that is delivered from a periphery of the ergodic relay is shown in FIG. 1A.

At 920, the method records photoacoustic signals detected by the ultrasonic array received through an output surface of the ergodic relay. The initial photoacoustic signals are encoded with time delays from the ergodic relay. The ultrasonic array or a DAQ (e.g., DAQ 380 in FIGS. 3 and 4) in electronic communication with an amplifier (e.g., amplifier 360 in FIGS. 3 and 4) in electronic communication with the ultrasonic array records the photoacoustic signals. For example, amplified radio frequency signals output from the amplifier can be recorded and digitized by the DAQ. The DAQ may record the radio frequency signals at time intervals defined by a sampling frequency.

In some cases, the recorded photoacoustic signals may be amplified. For example, an amplifier (e.g., amplifier 360 in FIGS. 3 and 4) in electronic communication with the ultrasonic array (e.g., ultrasonic array 340 in FIGS. 3 and 4) may amplify the recorded photoacoustic signals.

At 930, the method performs a time-reversal operation on the digitized photoacoustic waveforms acquired from the ultrasonic array to generate a set of time-reversed photoacoustic signals. In this context, time reversal comprises re-ordering each recorded acoustic waveform so that its final sample becomes the first and its first sample becomes the last. This time-reversal operation inverts the temporal evolution of the detected wavefield and therefore represents the wave propagation process in reverse. In some implementations, the photoacoustic signals detected by the ultrasonic array and digitized by the DAQ are stored in memory, numerically reversed in time, and optionally processed to compensate for system response, such as array impulse characteristics or relay dispersion. The resulting time-reversed digital waveforms are then converted back to analog radio-frequency signals by digital-to-analog output circuitry. These time-reversed signals are subsequently applied to the ultrasonic array so that the array re-emits a time-reversed acoustic field, which propagates backward through the ergodic relay toward the original photoacoustic source region and focuses energy at the locations where the initial photoacoustic emissions originated.

At 940, the method uses the ultrasonic array to emit the set of time-reversed photoacoustic signals into the ergodic relay through the output surface. In some cases, a set of amplified, time-reversed photoacoustic signals are emitted. In travelling between the output surface and the input surface, the time-reversed photoacoustic signals are transformed back into the initial photoacoustic signals, which are emitted into an acoustic medium such as water. When the acoustic waves hit the surface of the acoustic medium, the acoustic waves are reflected downward due to the high acoustic impedance mismatch at the interface between the air and the acoustic medium. A momentum change from the reflection of acoustic waves at the medium surface causes an acoustic radiation pressure pushing the surface upward inducing a surface displacement. As the time-reversed photoacoustic signals are emitted from the ultrasonic array into the ergodic relay, the acoustic radiation pressure from the reflections of the waves causes surface displacements that form a surface profile. The surface profile carries an imprint of the initial photoacoustic signal distribution from the object.

At 950, the method uses one or more light detectors (e.g., image sensors) of an optical imager to optically image the surface profile while the ultrasonic array transmits the set of time-reversed signals back into the ergodic relay to determine a set of image frames. For example, the light detector(s) may acquire image frames at a frame rate synchronized with the ultrasound pulses of the time-reversed photoacoustic signals emitted by the ultrasonic array. The image frames acquired may be used to determine the surface displacements defining the surface profile.

At 960, the method determines the displacements in the surface of the acoustic medium from the set of image frames to form a surface profile. In some implementations, profilometry can be used to detect the surface displacements. Some examples of profilometry techniques that can be used are described in Van der Jeught, S. and Dirckx, J. J., “Real-time structured light profilometry: a review, Optics and Lasers in Engineering 87, 18-31 (2016). For example, full-field profilometry techniques, such as structured-light projection or phase-shifting interferometry, may be used to recover the continuous surface height field across the imaged region. These methods analyze spatial fringes or projected patterns to extract local phase changes, which directly correspond to surface deflections induced by the acoustic waves.

At 970, the method maps the displacements along the surface profile to x-y locations to construct a 2D image of the object. Each displacement value reflects the local pressure-induced deformation of the acoustic-medium surface caused by photoacoustic waves emanating from the object. By assigning each displacement measurement to the correct pixel location in the x-y plane, the system generates a spatial distribution of acoustic energy that corresponds to optical absorption within the object. The resulting displacement map serves as a 2D photoacoustic projection image in which brighter or larger displacement values indicate regions of higher photoacoustic pressure generation.

At 980, the method optionally (denoted by dashed line) determines whether N images have been acquired by the optical imager. N can be 1, 2, 3, 4, 5, . . . 1000. According to one aspect, N can be in a range between 10-500.

If N images have not been acquired, the method uses a surface scanning device (e.g., surface scanning device 470 in FIG. 4) to move the surface of the acoustic medium to a new height (optional operation 982). If N images have been acquired, the method constructs a 3D image of the object by combining the 2D images (optional operation 990).

### Extension to Ultrasonic Therapy

In various embodiments, photoacoustic imaging systems generate time-reversed ultrasound signals (also referred to herein as time-reversed photoacoustic signals). Time-reversed ultrasound signals can be used in therapeutic applications, particularly for diseases associated with light absorbers, like blood. In photoacoustics, light absorption by chromophores (such as hemoglobin in blood) generates localized heating, which then produces acoustic waves. By leveraging this mechanism, the time-reversed ultrasound signals can be used to focus energy precisely on areas where light is absorbed, targeting specific tissues for thermal therapy or histotripsy. Some examples of thermal therapy are discussed in Maloney, E. & Hwang, J. H. “Emerging HIFU applications in cancer therapy,” International Journal of Hyperthermia 31, 302-309 (2015). Some examples of histotripsy are discussed in Williams, R. P., Simon, J. C., Khokhlova, V. A., Sapozhnikov, O. A. & Khokhlova, T. D., “The histotripsy spectrum: differences and similarities in techniques and instrumentation. International Journal of Hyperthermia 40, 2233720 (2023). Conditions like port wine stains could be treated by focusing the ultrasound energy on blood vessels, allowing for non-invasive targeting. Using time-reversed ultrasound signals in therapeutic applications could potentially lead to precise and non-invasive treatments, as the time-reversed ultrasound would focus therapeutic energy only on areas where light absorption generates strong acoustic signals, sparing surrounding tissues.

### IV. Computational Systems

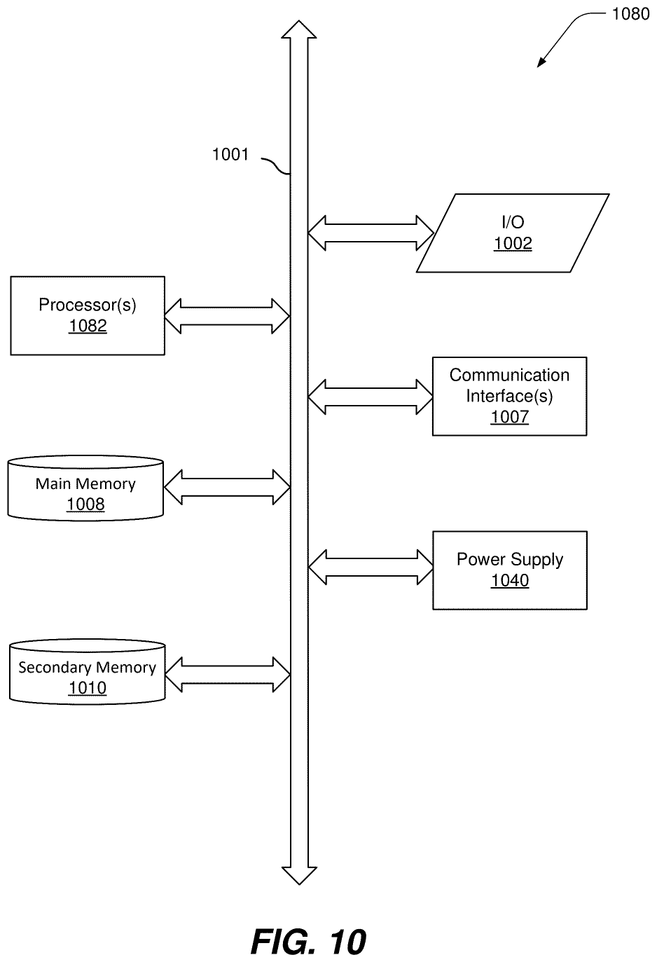

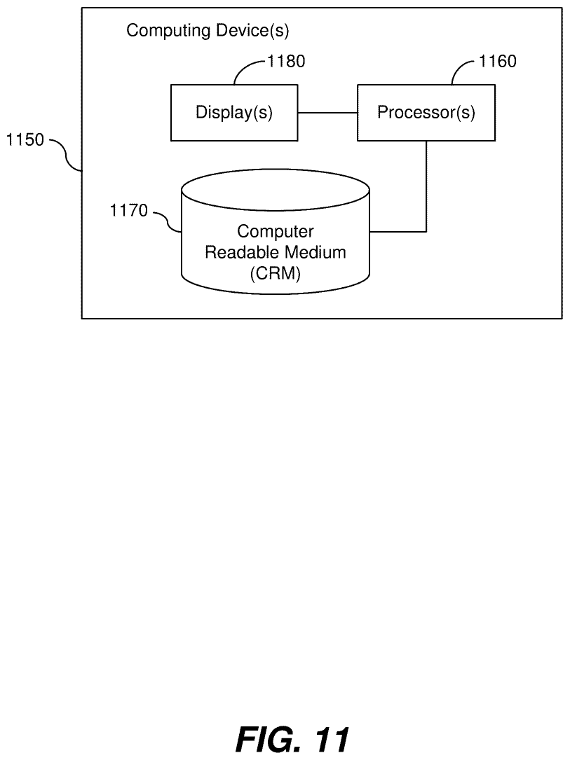

The techniques described above may be implemented using one or more computing devices. FIGS. 10 and 11 illustrate examples of computing devices that may be used, e.g., to implement functions of real-time surgical imaging methods described herein.

In FIG. 10, the computing device(s) 1080 includes a bus 1001 coupled to an input/output (I/O) subsystem 1002, one or more processors 1082, one or more communication interfaces 1007, a main memory 1008, a secondary memory 1010, and a power supply 1040. One or more of these components may be in separate housing. According to certain implementations, the illustrated components may be examples of components that are part of computing device 390 in FIGS. 3 and 4 or computing device 1190 in FIG. 11. Computing device 1080 includes I/O subsystem 1002, which includes, or is in communication with, one or more components which may implement an interface for interacting with human users and/or other computer devices depending upon the application.

Certain embodiments disclosed herein may be implemented in program code on computing device 1080 with I/O subsystem 1002 used to receive input program statements and/or data from a human user (e.g., via a graphical user interface (GUI), a keyboard, touchpad, etc.) and to display them back to the user, for example, on a display. The I/O subsystem 1002 may include, e.g., a keyboard, mouse, graphical user interface, touchscreen, or other interfaces for input, and, e.g., an LED or other flat screen display, or other interfaces for output. Other elements of embodiments may be implemented with a computer system like that of computer system 1000 without I/O subsystem 1002. According to various embodiments, a processor may include a CPU, GPU or computer, analog and/or digital input/output connections, controller boards, etc.

Program code may be stored in non-transitory computer readable media such as secondary memory 1010 or main memory 1008 or both. One or more processors 1004 may read program code from one or more non-transitory media and execute the code to enable computing device 1080 to accomplish the methods performed by various embodiments described herein, such as photoacoustic imaging methods. Those skilled in the art will understand that the one or more processors 1082 may accept source code and interpret or compile the source code into machine code that is understandable at the hardware gate level of the one or more processors 1082.

Communication interfaces 1007 may include any suitable components or circuitry used for communication using any suitable communication network (e.g., the Internet, an intranet, a wide-area network (WAN), a local-area network (LAN), a wireless network, a virtual private network (VPN), and/or any other suitable type of communication network). For example, communication interfaces 1007 can include network interface card circuitry, wireless communication circuitry, etc.

In certain embodiments, computing device 1080 may be part of or connected to a controller that is employed to control functions of various system components described herein. For example, computing device 1080 may control recording of signals by DAQ(s) or data acquisition by an ultrasonic transducer array or ultrasonic probe and/or delivery of energy an energy source. The controller will typically include one or more memory devices and one or more processors. The processor may include a CPU or computer, analog and/or digital input/output connections, stepper motor controller boards, etc.

In FIG. 11, the computing device(s) 1150 includes one or more processors 1160 (e.g., microprocessors), a non-transitory computer readable medium (CRM) 1170 in communication with the processor(s) 1160, and one or more displays 1180 also in communication with processor(s) 1160. Processor(s) 1160 is in electronic communication with CRM 1170 (e.g., memory). Processor(s) 1160 is also in electronic communication with display(s) 1180, e.g., to display image data, text, etc. on display 1180. Processor(s) 1160 may retrieve and execute instructions stored on the CRM 1170 to perform one or more functions described above. For example, processor(s) 1160 may execute instructions to perform one or more operations to time reverse photoacoustic signals, determine a surface profile of an acoustic medium, etc. The CRM (e.g., memory) 1170 can store instructions for performing one or more functions of the described above. These instructions may be executable by processor(s) 1160. CRM 1170 can also store raw images, e.g., microscopy images, sub-images sampled from an image, or the like.

Many types of computing devices having any of various computer architectures may be employed as the disclosed systems for implementing algorithms. For example, the computing devices may include software components executing on one or more general purpose processors or specially designed processors such as Application Specific Integrated Circuits (ASICs) or programmable logic devices (e.g., Field Programmable Gate Arrays (FPGAs)). Further, the systems may be implemented on a single device or distributed across multiple devices. The functions of the computational elements may be merged into one another or further split into multiple sub-modules.

At one level a software element is implemented as a set of commands prepared by the programmer/developer. However, the module software that can be executed by the computer hardware is executable code committed to memory using “machine codes” selected from the specific machine language instruction set, or “native instructions,” designed into the hardware processor. The machine language instruction set, or native instruction set, is known to, and essentially built into, the hardware processor(s). This is the “language” by which the system and application software communicates with the hardware processors. Each native instruction is a discrete code that is recognized by the processing architecture and that can specify particular registers for arithmetic, addressing, or control functions; particular memory locations or offsets; and particular addressing modes used to interpret operands. More complex operations are built up by combining these simple native instructions, which are executed sequentially, or as otherwise directed by control flow instructions.

The inter-relationship between the executable software instructions and the hardware processor is structural. In other words, the instructions per se are a series of symbols or numeric values. They do not intrinsically convey any information. It is the processor, which by design was preconfigured to interpret the symbols/numeric values, which imparts meaning to the instructions.

The algorithms used herein may be configured to execute on a single machine at a single location, on multiple machines at a single location, or on multiple machines at multiple locations. When multiple machines are employed, the individual machines may be tailored for their particular tasks. For example, operations requiring large blocks of code and/or significant processing capacity may be implemented on large and/or stationary machines.

In addition, certain embodiments relate to tangible and/or non-transitory computer readable media or computer program products that include program instructions and/or data (including data structures) for performing various computer-implemented operations. Examples of computer-readable media include, but are not limited to, memory devices, phase-change devices, magnetic media such as disk drives, magnetic tape, optical media such as CDs, magneto-optical media, and hardware devices that are specially configured to store and perform program instructions, such as read-only memory devices (ROM) and random access memory (RAM). The computer readable media may be directly controlled by an end user or the media may be indirectly controlled by the end user. Examples of directly controlled media include the media located at a user facility and/or media that are not shared with other entities. Examples of indirectly controlled media include media that is indirectly accessible to the user via an external network and/or via a service providing shared resources such as the “cloud.” Examples of program instructions include both machine code, such as produced by a compiler, and files containing higher level code that may be executed by the computer using an interpreter.

In some embodiments, code executed during generation or execution of various models on an appropriately programmed system can be embodied in the form of software elements which can be stored in a nonvolatile storage medium (such as optical disk, flash storage device, mobile hard disk, etc.), including a number of instructions for making a computing device (such as personal computers, servers, network equipment, etc.). In various embodiments, the data or information employed in the disclosed methods and apparatus is provided in an electronic format. Such data or information may include design layouts, fixed parameter values, floated parameter values, feature profiles, metrology results, and the like. As used herein, data or other information provided in electronic format is available for storage on a machine and transmission between machines. Conventionally, data in electronic format is provided digitally and may be stored as bits and/or bytes in various data structures, lists, databases, etc. The data may be embodied electronically, optically, etc.

### Example Embodiments

Embodiment 1: A photoacoustic imaging method, comprising: delivering, using at least one energy source, excitation energy into an object, the excitation energy inducing photoacoustic waves travelling through an ergodic relay in acoustic communication with the object via an acoustic medium; detecting, using an ultrasonic array, photoacoustic signals received through an output surface of the ergodic relay; time-reversing the photoacoustic signals detected by the ultrasonic array to generate a set of time-reversed photoacoustic signals; emitting, using the ultrasonic array, the set of time-reversed photoacoustic signals into the ergodic relay through the output surface; optically imaging a surface of the acoustic medium while the ultrasonic array emits the set of time-reversed photoacoustic signals to generate a surface profile of the acoustic medium; and reconstructing a two-dimensional (2D) image of the object from the surface profile.

Embodiment 2: The photoacoustic imaging method of embodiment 1, further comprising: scanning the surface of the acoustic medium to one or more additional heights; generating one or more additional surface profiles of the acoustic medium at the one or more additional heights; reconstructing one or more additional 2D images from the one or more additional surface profiles; and combining the 2D image with the one or more additional 2D images to generate a three-dimensional (3D) image of the object.

Embodiment 3: The photoacoustic imaging method of embodiment 1, further comprising amplifying the time-reversed photoacoustic signals.

Embodiment 4: The photoacoustic imaging method of embodiment 1, further comprising using profilometry to generate the surface profile.

Embodiment 5: The photoacoustic imaging method of embodiment 1, further comprising detecting displacements in the surface of the acoustic medium to generate the surface profile; and mapping the displacements to x-y locations within a field-of-view to reconstruct the 2D image.

Embodiment 7: A photoacoustic imaging system, comprising: an ergodic relay acoustically coupled to an acoustic medium; an ultrasonic array acoustically coupled to the ergodic relay, the ultrasonic array configured to detect photoacoustic signals received from the ergodic relay and emit time-reversed photoacoustic signals back into the ergodic relay; and an optical imager configured to detect one or more surface profiles of the acoustic medium to reconstruct one or more two-dimensional (2D) images of an object being imaged during operation.

Embodiment 8: The photoacoustic imaging system of embodiment 7, wherein the optical imager is configured to: optically image a plurality of surface profiles of a surface of the acoustic medium at different heights to reconstruct a plurality of two-dimensional (2D) images of the object; and combine the plurality of 2D images to construct a 3D image of the object.

Embodiment 9: The photoacoustic imaging system of embodiment 7, further comprising at least one energy source configured to provide excitation energy into the object being imaged.

Embodiment 10: The photoacoustic imaging system of embodiment 9, further comprising at least one energy source configured to provide the excitation energy into the object being imaged.

Embodiment 11: The photoacoustic imaging system of embodiment 9, wherein the at least one energy source comprises a laser source.

Embodiment 12: The photoacoustic imaging system of embodiment 7, wherein the ergodic relay includes a receptacle for receiving the acoustic medium.

Embodiment 13: The photoacoustic imaging system of embodiment 12, wherein the receptacle comprises a volume configured to receive at least a portion of the object being imaged.

Embodiment 14: The photoacoustic imaging system of embodiment 12, wherein the receptacle includes a volume with a curved input surface configured to receive photoacoustic waves from the object being imaged.

Embodiment 15: The photoacoustic imaging system of embodiment 14, wherein the curved input surface has a hemispherical shape.

Embodiment 16: The photoacoustic imaging system of embodiment 7, wherein the ultrasonic array is a 0D ultrasonic array, a 1D ultrasonic array, or a 2D ultrasonic array.

Embodiment 17: A non-transitory machine-readable medium comprising instructions that, when executed by one or more processors, are configured to cause the one or more processors to perform operations comprising: causing transmission of excitation energy into an object; recording photoacoustic signals detected by the ultrasonic array through an output surface of the ergodic relay; time-reversing the photoacoustic signals detected by an ultrasonic array to generate a set of time-reversed photoacoustic signals; causing, using the ultrasonic array, emission of the set of time-reversed photoacoustic signals into the ergodic relay through the output surface; causing an optical imager to image a surface of the acoustic medium while the ultrasonic array emits the set of time-reversed photoacoustic signals to generate a surface profile of the acoustic medium; and reconstructing a two-dimensional (2D) image of the object from the surface profile.

Embodiment 18: The non-transitory machine-readable medium of embodiment 17, further comprising: scanning the surface of the acoustic medium to one or more additional heights; generating one or more additional surface profiles of the acoustic medium at the one or more additional heights; reconstructing one or more additional 2D images from the one or more additional surface profiles; and combining the 2D image with the one or more additional 2D images to generate a three-dimensional (3D) image of the object.

Embodiment 19: The non-transitory machine-readable medium of embodiment 17, further comprising amplifying the time-reversed photoacoustic signals.

Embodiment 20: The non-transitory machine-readable medium of embodiment 17, further comprising using profilometry to generate the surface profile.

Embodiment 21: The non-transitory machine-readable medium of embodiment 17, further comprising: detecting a plurality of displacements in the surface of the acoustic medium to generate the surface profile; and mapping the displacements to x-y locations within a field-of-view to reconstruct the 2D image.

Embodiment 22: The non-transitory machine-readable medium of embodiment 17, further comprising using profilometry to generate the surface profile.

Modifications, additions, or omissions may be made to any of the above-described embodiments without departing from the scope of the disclosure. Any of the embodiments described above may include more, fewer, or other features without departing from the scope of the disclosure. Additionally, the steps of described features may be performed in any suitable order without departing from the scope of the disclosure. Also, one or more features from any embodiment may be combined with one or more features of any other embodiment without departing from the scope of the disclosure. The components of any embodiment may be integrated or separated according to particular needs without departing from the scope of the disclosure.

It should be understood that certain aspects described above can be implemented in the form of logic using computer software in a modular or integrated manner. Based on the disclosure and teachings provided herein, a person of ordinary skill in the art will know and appreciate other ways and/or methods to implement the present invention using hardware and a combination of hardware and software.

Any of the software components or functions described in this application, may be implemented as software code using any suitable computer language and/or computational software such as, for example, Java, C, C#, C++ or Python, LabVIEW, Mathematica, or other suitable language/computational software, including low level code, including code written for field programmable gate arrays, for example in VHDL. The code may include software libraries for functions like data acquisition and control, motion control, image acquisition and display, etc. Some or all of the code may also run on a personal computer, single board computer, embedded controller, microcontroller, digital signal processor, field programmable gate array and/or any combination thereof or any similar computation device and/or logic device(s). The software code may be stored as a series of instructions, or commands on a CRM such as a random access memory (RAM), a read only memory (ROM), a magnetic media such as a hard-drive or a floppy disk, or an optical media such as a CD-ROM, or solid stage storage such as a solid state hard drive or removable flash memory device or any suitable storage device. Any such CRM may reside on or within a single computational apparatus, and may be present on or within different computational apparatuses within a system or network. Although the foregoing disclosed embodiments have been described in some detail to facilitate understanding, the described embodiments are to be considered illustrative and not limiting. It will be apparent to one of ordinary skill in the art that certain changes and modifications can be practiced within the scope of the appended claims.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

What is claimed is:

1. A photoacoustic imaging method, comprising:
delivering, using at least one energy source, excitation energy into an object, the excitation energy inducing photoacoustic waves travelling through an ergodic relay in acoustic communication with the object via an acoustic medium;
detecting, using an ultrasonic array, photoacoustic signals received through an output surface of the ergodic relay;
time-reversing the photoacoustic signals detected by the ultrasonic array to generate a set of time-reversed photoacoustic signals;
emitting, using the ultrasonic array, the set of time-reversed photoacoustic signals into the ergodic relay through the output surface;
optically imaging a surface of the acoustic medium while the ultrasonic array emits the set of time-reversed photoacoustic signals to generate a surface profile of the acoustic medium; and
reconstructing a two-dimensional (2D) image of the object from the surface profile.

2. The photoacoustic imaging method of claim 1, further comprising:
scanning the surface of the acoustic medium to one or more additional heights;
generating one or more additional surface profiles of the acoustic medium at the one or more additional heights;
reconstructing one or more additional 2D images from the one or more additional surface profiles; and
combining the 2D image with the one or more additional 2D images to generate a three-dimensional (3D) image of the object.

3. The photoacoustic imaging method of claim 1, further comprising amplifying the time-reversed photoacoustic signals.

4. The photoacoustic imaging method of claim 1, further comprising using profilometry to generate the surface profile.

5. The photoacoustic imaging method of claim 1, further comprising:
detecting displacements in the surface of the acoustic medium to generate the surface profile; and
mapping the displacements to x-y locations within a field-of-view to reconstruct the 2D image.

6. A photoacoustic imaging system, comprising:
an ergodic relay acoustically coupled to an acoustic medium;
an ultrasonic array acoustically coupled to the ergodic relay, the ultrasonic array configured to detect photoacoustic signals received from the ergodic relay and emit time-reversed photoacoustic signals back into the ergodic relay; and
an optical imager configured to detect one or more surface profiles of the acoustic medium to reconstruct one or more two-dimensional (2D) images of an object being imaged during operation.

7. The photoacoustic imaging system of claim 6, wherein the optical imager is configured to:
optically image a plurality of surface profiles of a surface of the acoustic medium at different heights to reconstruct a plurality of two-dimensional (2D) images of the object; and
combine the plurality of 2D images to construct a 3D image of the object.

8. The photoacoustic imaging system of claim 6, further comprising at least one energy source configured to provide excitation energy into the object being imaged.

9. The photoacoustic imaging system of claim 8, further comprising at least one energy source configured to provide the excitation energy into the object being imaged.

10. The photoacoustic imaging system of claim 8, wherein the at least one energy source comprises a laser source.

11. The photoacoustic imaging system of claim 6, wherein the ergodic relay includes a receptacle for receiving the acoustic medium.

12. The photoacoustic imaging system of claim 11, wherein the receptacle comprises a volume configured to receive at least a portion of the object being imaged.

13. The photoacoustic imaging system of claim 11, wherein the receptacle includes a volume with a curved input surface configured to receive photoacoustic waves from the object being imaged.

14. The photoacoustic imaging system of claim 13, wherein the curved input surface has a hemispherical shape.

15. The photoacoustic imaging system of claim 6, wherein the ultrasonic array is a 0D ultrasonic array, a 1D ultrasonic array, or a 2D ultrasonic array.

16. A non-transitory machine-readable medium comprising instructions that, when executed by one or more processors, are configured to cause the one or more processors to perform operations comprising:
causing transmission of excitation energy into an object, the excitation energy inducing photoacoustic waves travelling through an ergodic relay in acoustic communication with the object via an acoustic medium;
recording photoacoustic signals detected by an ultrasonic array through an output surface of the ergodic relay;
time-reversing the photoacoustic signals detected by the ultrasonic array to generate a set of time-reversed photoacoustic signals;
causing, using the ultrasonic array, emission of the set of time-reversed photoacoustic signals into the ergodic relay through the output surface;
causing an optical imager to image a surface of the acoustic medium while the ultrasonic array emits the set of time-reversed photoacoustic signals to generate a surface profile of the acoustic medium; and
reconstructing a two-dimensional (2D) image of the object from the surface profile.

17. The non-transitory machine-readable medium of claim 16, further comprising:
scanning the surface of the acoustic medium to one or more additional heights;
generating one or more additional surface profiles of the acoustic medium at the one or more additional heights;
reconstructing one or more additional 2D images from the one or more additional surface profiles; and
combining the 2D image with the one or more additional 2D images to generate a three-dimensional (3D) image of the object.

18. The non-transitory machine-readable medium of claim 16, further comprising amplifying the time-reversed photoacoustic signals.

19. The non-transitory machine-readable medium of claim 16, further comprising using profilometry to generate the surface profile.

20. The non-transitory machine-readable medium of claim 16, further comprising:
detecting a plurality of displacements in the surface of the acoustic medium to generate the surface profile; and
mapping the displacements to x-y locations within a field-of-view to reconstruct the 2D image.
